(19) United States
(12) Patent Application Publication
(10) Pub. No.: US 2005/0196458 A1
(43) Pub. Date: Sep. 8, 2005

(54) Odor absorbing pad for shoes

(76) Inventor: Jaime Valenzuela, Los Angeles, CA (US)

Correspondence Address: TOPE-MCKAY & ASSOCIATES, 23852 PACIFIC COAST HIGHWAY #311, MALIBU, CA, 90265, US

(21) Appl. No.: 11/070,042

(22) Filed: Mar. 2, 2005

Related U.S. Application Data
(60) Provisional application No. 60/549,650, filed on Mar. 2, 2004.

(51) Int. Cl. B65D 88/54; A61K 9/14

(52) U.S. Cl. 424/489; 222/310

(57) ABSTRACT

The present invention relates to odor absorbing pad for shoes. More particularly, it relates to odor absorbing pad having a powder dispersion system. The powder dispersion system has a first housing connected with a second housing, where the two housings are configured to contain a powder delivery medium with a specific powder. When at least one housing is actuated, the powder delivery medium causes the specific powder to pass through a moisture absorbent portion into a foot compartment of a shoe where it is distributed about a foot.

### PRIORITY CLAIM

This application is a non-provisional application, claiming the benefit of priority to provisional application No. 60/549,650, filed in the United States on Mar. 2, 2004, and entitled “Odor Absorbing Pad for Shoes.”

### FIELD OF INVENTION

The present invention relates to an odor absorbing pad for shoes and more particularly, to a powder dispersion system for dispersing powder throughout a shoe compartment and removing odor from the shoe.

### BACKGROUND OF INVENTION

The use of shoe liners is known in prior art. More specifically, shoe liners heretofore devised and utilized for a purpose of providing comfort to a user and removing odor are known to consist basically of familiar, expected and obvious structural configurations. By way of example, the prior art includes U.S. Pat. No. 5,399,404; U.S. Pat. No. 4,864,740; U.S. Pat. No. 4,257,176; U.S. Pat. No. 4,185,402; and U.S. Pat. No. 5,261,169.

Odor absorbing pad often contain a desired specific powder. The specific powder may be an anti-fungal powder, an anti-odor powder, a medicinal powder, or a scented powder. Although the specific powder will serve its purpose if released from the odor-absorbing pad, it is often trapped therein. The odor absorbing pad previously devised includes no means of kneading the specific powder and thereby forcing it into a foot compartment of a shoe. Additionally, previously devised odor absorbing pad do not allow for ease of refilling, often resulting in powder depletion. Furthermore, pads placed in a heel alone do little to spread the specific powder throughout the entirety of the shoe.

Therefore, it can be appreciated that there exists a continuing need for a new and improved odor absorbing pad for shoes with a device to actuate and release a specific powder about a shoe. In this regard, the present invention substantially fulfills this need.

### SUMMARY OF INVENTION

The present invention relates to a powder dispersion system for dispersing powder into a foot compartment of a shoe. The powder dispersion system comprises a first housing having an interior, an exterior, a first side and a second side, with holes in the first side passing between the interior and the exterior; and a second housing having an interior, an exterior, a first side and a second side, with holes in the first side passing between the interior and the exterior. The interior of the second housing is fluidly connected with the interior of the first housing. Additionally, the first and second housings are configured to contain a specific powder and are further configured to be connected with a bottom portion of a shoe, whereby when the powder dispersion system is actuated, the specific powder is passed from the powder dispersion system into a foot compartment of a shoe where it is distributed about a foot.

In actual use, walking kneads the contents of the powder dispersion system and slowly forces the specific powder from the system and into the foot compartment of a shoe. Upon powder depletion, it may be desirable to refill the powder dispersion system. The system further comprises a filler mechanism, allowing a user to lift an insole of the shoe and refill the system through use of the filler mechanism. The filler mechanism is a filler hole with a detachably attached filler plug. When refilling a housing embedded in the sole of the shoe, the powder travels through the housing connector to a housing embedded in a toe portion of the shoe, thereby refilling the entire powder dispersion system.

In another aspect, the holes are micro-holes, and are configured to be in fluid communication with a moisture absorbent portion. The moisture absorbent portion is a shoe insert.

In yet another aspect, the moisture absorbent portion is attached above the first sides of the housing, whereby the moisture absorbent portion reduces an amount of moisture penetrating into the powder dispersion system.

In another aspect, the moisture absorbent portion has at least one aperture, with the at least one aperture allowing free passage of air and powder from the powder dispersion system into the foot compartment of a shoe.

Additionally, the moisture absorbent portion is filled with a highly absorbent material surrounded by a hydrophobic material in order to absorb and retain a substantial part of any moisture generated by the foot.

Furthermore, the first and second housings are elasticly deformable.

In another aspect, the present invention further comprises a kneading device connected with the bottom side of each housing. The kneading device is selected from a group consisting of a spring and at least one raised portion.

The present invention further comprises both a powder delivery medium and a specific powder included within the interior of the first and second portions. The powder delivery medium is selected from a group consisting of charcoal and baking soda. Additionally, the specific powder includes a powder selected from a group consisting of an anti-fungal powder, an anti-odor powder, a medicinal powder, and a scented powder.

Furthermore, the first housing is constructed of plastic and comprises a plurality of compartments.

In another aspect, any compartment is interchangeable with any other compartment in the plurality of compartments, where each compartment contains a powder delivery medium with a specific powder, and where the specific powder of a compartment is different from the specific powder of other compartments, where a compartment with a specific powder content may be placed in the first housing at any position.

In yet another aspect, the powder dispersion system is formed such that it is detachably attachable with a sole of a shoe or such that it is permanently attachable with a sole of a shoe.

As can be appreciated by one in the art, the present invention also comprises a method for forming the odor absorbing pad described herein. The method comprising acts of forming the respective parts of the present invention.

### BRIEF DESCRIPTION OF THE DRAWINGS

The nature of the odor absorbing pad described herein will be readily apparent in the following drawings, in which:

FIG. 1A is a cross-sectional view of the present invention, illustrating each section of a powder dispersion system;

FIG. 1B is a top view of a powder dispersion system according to the present invention;

FIG. 2 is a side of the present invention, illustrating an inter-relation between sections;

FIG. 3A is a top view of the present invention, illustrating a moisture absorbent portion with a powder dispersion system attached thereto;

FIG. 3B is a side view of the present invention, illustrating the moisture absorbent portion with the powder dispersion system attached thereto;

FIG. 4 is a top view of the present invention, showing the powder dispersion system positioned within a shoe; and

FIG. 5 is a side view of the present invention, showing the powder dispersion system positioned within the shoe.

### DETAILED DESCRIPTION

The present invention relates to an odor absorbing pad for shoes. More particularly, it relates to an odor absorbing pad having a powder dispersion system. The powder dispersion system has a first housing connected with a second housing, where the two housings are configured to contain a powder delivery medium with a specific powder. When at least one housing is actuated, the powder delivery medium causes the specific powder to pass through a moisture absorbent portion into a foot compartment of a shoe where it is distributed about a foot.

The following description, taken in conjunction with the referenced drawings, is presented to enable one of ordinary skill in the art to make and use the invention. Various modifications will be readily apparent to those skilled in the art, and the general principles defined herein may be applied to a wide range of aspects. Thus, the present invention is not intended to be limited to the aspects presented, but is to be accorded the widest scope consistent with the principles and novel features disclosed herein. Furthermore it should be noted that unless explicitly stated otherwise, the figures included herein are illustrated diagrammatically and without any specific scale, as they are provided as qualitative illustrations of the concept of the present invention.

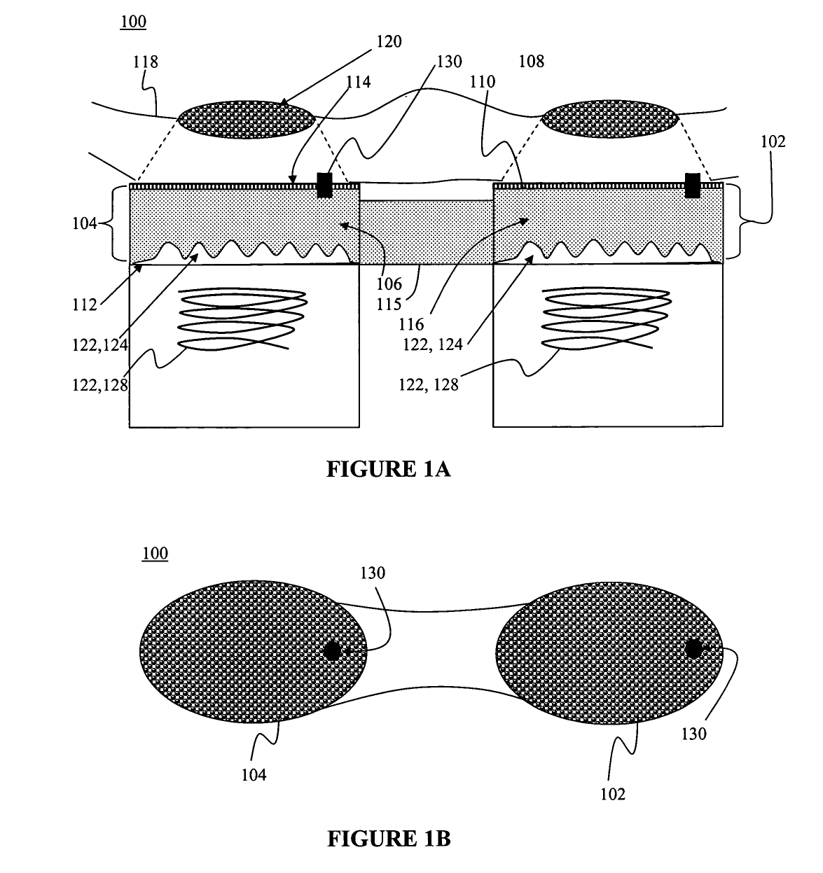

Referring to the figures, FIG. 1A illustrates an aspect of the powder dispersion system 100 in accordance with the present invention. The powder dispersion system 100 has a first housing 102 and a second housing 104, with at least one if not both being elastically deformable. Each of the first 102 and second 104 housings have an interior 106, an exterior 108, a first side 110, and a second side 112. The second sides 112 include holes 114 passing from the interior 106 to the exterior 108. The holes 114 may be any suitable size or shape, such as micro-holes. The interior 106 of the first housing 102 is fluidly connected with the interior 106 of the second housing 104 through a housing connector 115. Both the first 102 and second 104 housings may be any suitable shape. For example, from a top view, the housings 102 and 104 may be substantially circular in shape, or from a side perspective view, may be rectangular. Additionally, the housings 102 and 104 may be constructed of any suitable material, a non-limiting example of which includes plastic and polyurethane.

Furthermore, the housings 102 and 104 are configured to contain a powder delivery medium mixed with a specific powder 116. The powder delivery medium may be any suitable medium for delivering a powder, non-limiting examples of which include charcoal and baking soda. The specific powder may be any powder providing a desirable result, non-limiting examples of which include anti-fungal powder, an anti-odor powder, a medicinal powder, and a scented powder.

The holes 114 are configured to be in fluid communication with a moisture absorbent portion 118, such as an insertable shoe insole. The moisture absorbent portion 118 may be constructed of any suitable material, a non-limiting example of which includes being filled with a highly absorbent material surrounded by a hydrophobic material in order to absorb and retain a substantial part of any moisture generated by a foot. Furthermore, the moisture absorbent portion 118 has at least one aperture 120. The moisture absorbent portion 118 is attached above the first sides 110 of the housings 102 and 104 such that the at least one aperture 120 is aligned with the holes 114, allowing passage of air and powder from the powder dispersion system 100 into the foot compartment of a shoe.

Additionally, the housings 102 and 104 may contain a plurality of manually interchangeable compartments. Each compartment may contain the powder delivery medium with the specific powder, where the specific powder component of one compartment may be different from the specific powder of other apartments.

Connected with the bottom side 118 of at least one of the housings 102 and 104 is a kneading device 122. The kneading device 122 may be any suitable device for kneading the housings 102 and 104, non-limiting examples of which include a spring 124 and at least one raised portion 128. For example, when the kneading device 122 is a spring 124, the spring may be embedded in a sole of shoe to knead the bottom side 118 of at least one of the housings 102 and 104. Or alternatively, when the kneading device 122 is at least one raised portion 128, the raised portion kneads any powder delivery medium and specific powder 116 included within the interior 106 of the housings 102 and 104. Through use of the kneading device 122, the powder delivery medium and specific powder 116 are kneaded and pushed through the holes 114.

Furthermore, the powder dispersion system 100 may include a filler mechanism 130 in at least one of the housings 102 and 104. The filler mechanism 130 may be any suitable mechanism or device allowing a user to add the powder delivery medium and specific powder 116 to the powder dispersion system 100, a non-limiting example of which includes a filler hole and a detachably attached filler plug. Through use of the filler mechanism 130, a user may refill the system 100 as needed. The filler mechanism may be placed at any suitable location for ease of access. For example, the filler mechanism may be included in the first side 110 of the housings 102 and 104, allowing a user to remove the moisture absorbent portion 118 (insertable shoe insole), remove the filler plug and re-fill the housings 102 and 104.

Illustrated in FIG. 1B is a top view of the powder dispersion system 100. As shown in this perspective, the powder dispersion system 100 may be substantially circular in shape. Additionally, the filler mechanism 130 of the housings 102 and 104 is accessible from the first side 110 of the powder dispersion system 100.

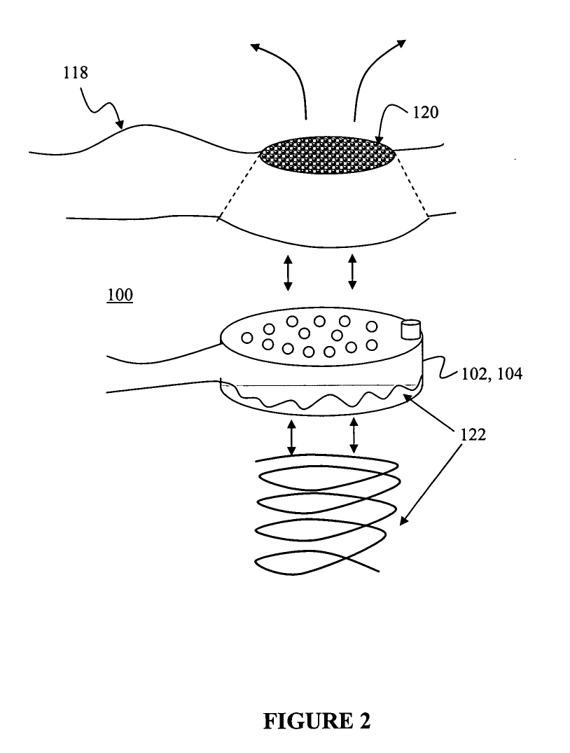

FIG. 2 illustrates the interrelation between each section. As shown in FIG. 2, the kneading device 122, the elastically deformable housings 102 and 104, and the moisture absorbent portion 118 are configured to generate airflow through the powder dispersion system 100, thereby preventing moisture from penetrating into the elastically deformable housing 114. When the powder dispersion system 100 is actuated, the kneading device 122 moves the elastically deformable housings 102 and 104, causing the powder delivery medium to release a specific powder. The specific powder is then passed from the elastically deformable housings 102 and 104 and through the micro-holes 114. The specific powder then passes through the at least one aperture 120 into a foot compartment of a shoe where it is distributed about a foot.

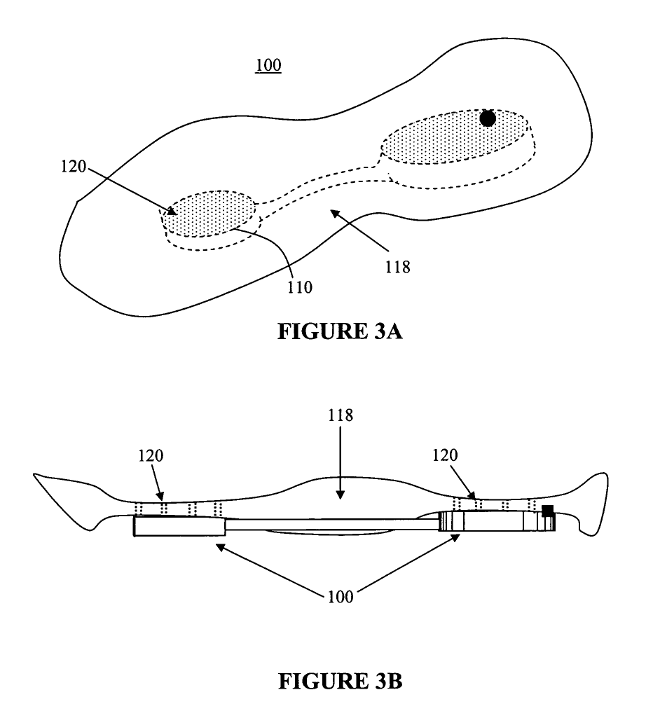

Illustrated in FIG. 3A is a powder dispersion system 100. As shown in this aspect, a moisture absorbent portion 118 is in communication with the first side 110 of each of the housings 102 and 104, thereby reducing an amount of moisture from penetrating into the powder dispersion system 100. The moisture absorbent portion 118 has at least one aperture 120, allowing free passage of air and specific powder from the dispersion system 100 into the foot compartment of a shoe.

Illustrated in FIG. 3B is a side perspective view of the moisture absorbent portion 118 connected with a powder dispersion system 100. As shown, the powder dispersion system 100 is in fluid communication with the at least one apertures 120, allowing the specific powder to pass from the powder dispersion systems 100, through the at least one aperture 120 and into the foot compartment of the shoe.

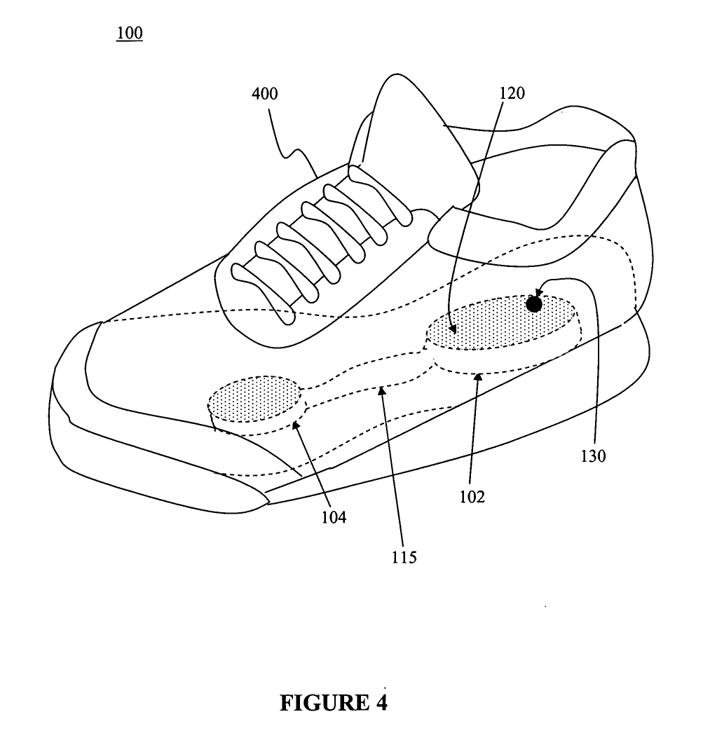

Illustrated in FIG. 4 is a powder dispersion system 100 placed within a shoe 400. The powder dispersion system 100 is formed such that it may be detachably attachable with a sole of the shoe 400, or may be permanently affixed and embedded within the sole of the shoe 400.

In actual use, when the system 100 is embedded within the sole of the shoe 400, walking kneads the contents of the powder dispersion system 100 and forces the specific powder from the system 100, through the at least one aperture 120 and into the foot compartment of a shoe. As the specific powder becomes depleted, it may be desirable to add additional specific powder. Through raising the moisture absorbent portion (shoe insole), the filler mechanism 130 is exposed, thereby allowing a user to utilize the filler mechanism to refill the powder dispersion system 100. Through use of the housing connector 115, specific powder inserted into the first housing 102 travels through the housing connector 115 to refill the second housing 104.

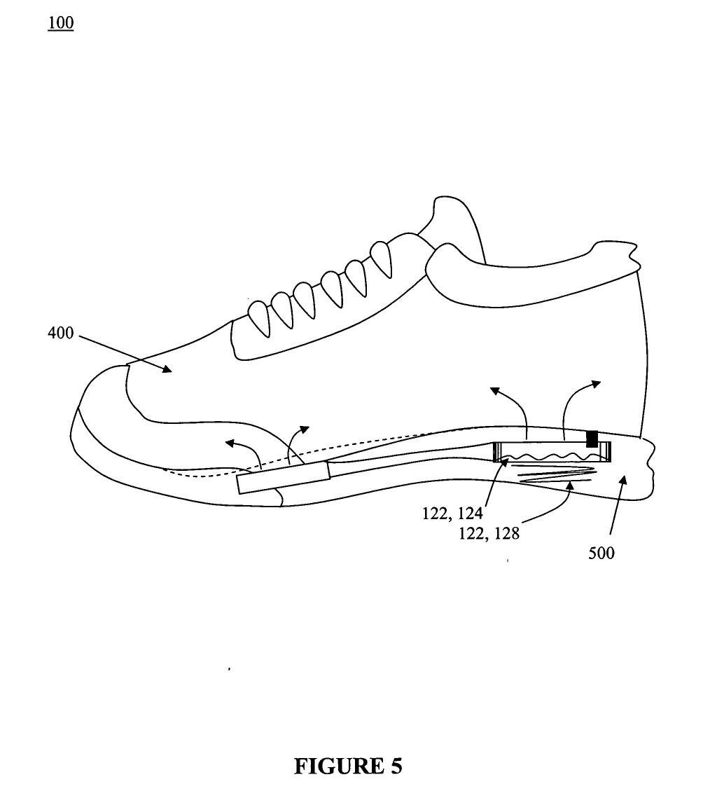

Illustrated in FIG. 5 is a side view, showing the powder dispersion system 100 embedded within a sole 500 of the shoe 400. As shown, the kneading device 122 may be at least one raised portion 124 or a spring 128. When a spring 128, the spring 128 is also embedded within the sole 500. The spring 128 is positioned such that walking actuates the spring 128, causing the spring to knead a portion of at least one of the housings 102 and 104.

In another aspect and as can be appreciated by on in the art, the odor absorbing pad described herein can be incorporated entirely into a shoe insole (i.e., insert), where all of the components of the odor absorbing pad are formed to fit within and be part of the shoe insole. When incorporated into an insole, the odor absorbing pad can be positioned within any existing shoe, allowing for versatility and utility in a wide variety of shoes. In this aspect, when desirable, the insole (i.e., odor absorbing pad) may be replaced with another odor absorbing pad.

1. A powder dispersion system for dispersing powder into a foot compartment of a shoe, the powder dispersion system comprising:
a first housing having an interior, an exterior, a first side and a second side, with holes in the first side passing between the interior and the exterior;
a second housing having an interior, an exterior, a first side and a second side, with holes in the first side passing between the interior and the exterior, the interior of the second housing being fluidly connected with the interior of the first housing, and where the first and second housings are configured to contain a specific powder and are further configured to be connected with a bottom portion of a shoe, whereby when the powder dispersion system is actuated, the specific powder is passed from the powder dispersion system into a foot compartment of a shoe where it is distributed about a foot.

2. A powder dispersion system as set forth in claim 1, wherein the holes are micro-holes.

3. A powder dispersion system as set forth in claim 1, wherein the holes are configured to be in fluid communication with a moisture absorbent portion.

4. A powder dispersion system as set forth in claim 3, wherein the moisture absorbent portion is a shoe insert.

5. A powder dispersion system as set forth in claim 3, where the moisture absorbent portion is attached above the first sides of the housing, whereby the moisture absorbent portion reduces an amount of moisture penetrating into the powder dispersion system.

6. A powder dispersion system as set forth in claim 3, where the moisture absorbent portion has at least one aperture, the at least one aperture allowing free passage of air and powder from the powder dispersion system into the foot compartment of a shoe.

7. A powder dispersion system as set forth in claim 3, wherein the moisture absorbent portion is filled with a highly absorbent material surrounded by a hydrophobic material in order to absorb and retain a substantial part of any moisture generated by the foot.

8. A powder dispersion system as set forth in claim 1, wherein the first and second housings are elasticly deformable.

9. A powder dispersion system as set forth in claim 1, further comprising an kneading device connected with the bottom side of each housing.

10. A powder dispersion system as set forth in claim 9, wherein the kneading device is a spring.

11. A powder dispersion system as set forth in claim 9, wherein the kneading device is at least one raised portion.

12. A powder dispersion system as set forth in claim 1, further comprising both a powder delivery medium and a specific powder included within the interior of the first and second portions.

13. A powder dispersion system as set forth in claim 12, wherein the powder delivery medium is selected from a group consisting of charcoal and baking soda.

14. A powder dispersion system as set forth in claim 1, wherein the specific powder includes a powder selected from a group consisting of an anti-fungal powder, an anti-odor powder, a medicinal powder, and a scented powder.

15. A powder dispersion system as set forth in claim 1, wherein the first housing is constructed of plastic and comprises a plurality of compartments.

16. A powder dispersion system as set forth in claim 15, wherein any compartment is interchangeable with any other compartment in the plurality of compartments, and where each compartment contains a powder delivery medium with a specific powder, where the specific powder of a compartment is different from the specific powder of other compartments, and where a compartment with a specific powder content may be placed in the first housing at any position.

17. A powder dispersion system as set forth in claim 1, wherein the powder dispersion system is formed such that it is detachably attachable with a sole of a shoe or such that it is permanently attachable with a sole of a shoe.

18. A powder dispersion system as set forth in claim 1, further comprising a filler mechanism in at least one housing, thereby allowing a user to add specific powder to the powder dispersion system.

19. A powder dispersion system as set forth in claim 18, wherein the filler mechanism is a filler hole with a detachably attached filler plug.

20. A method for forming a powder dispersion system for dispersing powder into a foot compartment of a shoe, the method comprising acts of:
forming a first housing having an interior, an exterior, a first side and a second side, with holes in the first side passing between the interior and the exterior;
forming a second housing having an interior, an exterior, a first side and a second side, with holes in the first side passing between the interior and the exterior, the interior of the second housing being fluidly connected with the interior of the first housing, and where the first and second housings are configured to contain a specific powder and are further configured to be connected with a bottom portion of a shoe, whereby when the powder dispersion system is actuated, the specific powder is passed from the powder dispersion system into a foot compartment of a shoe where it is distributed about a foot.
